(19) United States
(12) Patent Application Publication
(10) Pub. No.: US 2025/0065097 A1
(43) Pub. Date: Feb. 27, 2025

(54) VACUUM LUER LOCK FOR MEDICAL INFUSION FLUID LINES CONNECTORS

(71) Applicant: CareFusion 303, Inc., San Diego, CA (US)

(72) Inventor: Shahriar Shams, Whittier, CA (US)

(21) Appl. No.: 18/808,683

(22) Filed: Aug. 19, 2024

Related U.S. Application Data
(60) Provisional application No. 63/578,031, filed on Aug. 22, 2023.

(51) Int. Cl. A61M 39/10; A61M 39/00; A61M 39/24

(52) U.S. Cl. CPC A61M 39/1011; A61M 39/24; A61M 2039/0009; A61M 2039/1027; A61M 2205/583

(57) ABSTRACT

Aspects of the present disclosure describe a vacuum luer lock connector comprising a male luer configured to couple to a female luer defining a fluid passageway, a vacuum chamber between the male luer and female luer that entraps air when the male luer is pushed into the female luer, a solid cylinder that surrounds the male luer and acts as a piston, a cylindrical member that surrounds the female luer and acts as the walls of the vacuum chamber and a vent passage in the female luer that allows air to escape when the male luer is pushed into the female luer. The male and female luers are held connected by a force created in the vacuum chamber that surrounds the luers.

### CROSS REFERENCE TO RELATED APPLICATION

This application claims the benefit of priority under 35 U.S.C. § 119 to U.S. Provisional Patent Application Ser. No. 63/578,031, entitled “VACUUM LUER LOCK FOR MEDICAL INFUSION FLUID LINES CONNECTORS”, filed on Aug. 22, 2023, the entire contents of which is incorporated herein by reference.

### TECHNICAL FIELD

The present disclosure generally relates to medical infusion fluid lines connectors, and more particularly to a vacuum luer lock connector. This vacuum luer lock connection is held connected by a vacuum force that is generated in a vacuum chamber that surrounds the male and female luers of the connector. The connection can easily be disconnected by allowing outside air to rush into the vacuum chamber through designed vent passages.

### BACKGROUND

Patients are commonly injected with IV solutions that are initially provided in an IV reservoir (a bottle or bag) and dripped into the vein of the patient through an IV line. Typically, an injection port is provided along the IV line and adapted to function with a syringe to permit an injectate to be added to the IV solution. Medical infusion fluid lines connectors dedicate a safe pathway for aspiration of fluids, medications, or blood draws via a peripheral, arterial, or central venous catheter.

The conventional luer lock connector relies on the friction between the two tapered luers that are pushed against each other by the mechanical advantages of taper, and screw mechanism (luer nut). This results in a high contact force over a large area of contact for sealing purposes which also result in difficulties for the user to disconnect the luer. On the other hand, some fluids like blood, alcohol, and some medications may get dry between the two connections and result in bonding the two contacting surfaces. This further complicates the disconnection process. This type of connection is easy to connect but very difficult to disconnect since there is no control over the forces involved.

### SUMMARY

The present disclosure generally relates to luer connections, and more particularly to a vacuum luer lock connector.

In accordance with various embodiments of the present disclosure, a vacuum luer lock connector comprises a male luer configured to couple to a female luer defining a fluid passageway, a vacuum chamber between the male luer and female luer that entraps air when the male luer is pushed into the female luer, a solid cylinder that surrounds the male luer and acts as a piston, a cylindrical member that surrounds the female luer and acts as the walls of the vacuum chamber and a vent passage in the female luer that allows air to escape when the male luer is pushed into the female luer. The male and female luers are held connected by a force created in the vacuum chamber that surrounds the luers.

Embodiments of the present disclosure provide a vacuum luer lock connector, comprising a male luer configured to couple to a female luer defining a fluid passageway, a vacuum chamber between the male luer and female luer that entraps air when the male luer is pushed into the female luer, wherein the male luer and female luer are held connected by a force created in the vacuum chamber that surrounds the male luer and female luer.

In some embodiments, the vacuum luer lock connector further comprises a vent passage in the female luer that allows air to escape when the male luer is pushed into the female luer. In some embodiments, the vacuum luer lock connector further comprises a piston, wherein the piston is a solid cylinder that surrounds the male luer. In some embodiments, the vacuum luer lock connector further comprises a cylindrical member that surrounds the female luer and acts as a wall of the vacuum chamber. In some embodiments, the vacuum luer lock connector further comprises an o-ring installed inside the female luer that prevents an infusion fluid from leaking into the vacuum chamber. In some embodiments, a plurality of o-rings are installed inside the female luer.

In some embodiments, the vacuum luer lock connector further comprises a check valve on an outer surface of the vent passage that prevent the air from flowing back into the vacuum chamber. In some embodiments, the vacuum luer lock connector further comprises a second vent passage in the piston of the male luer that connects a compression area of the male luer to a surface of the male luer that mates with the female luer, the surface being perpendicular to a longitudinal axis of the male luer, and a slot created on an inside surface of the cylindrical member, the slot extending from an edge of the cylindrical member that mates with the male luer in a direction away from the male luer along a longitudinal axis of the solid cylinder, wherein a vacuum seal is maintained when the second vent passage and the slot are not aligned with one another along a longitudinal axis of the vacuum luer lock connector. In some embodiments, when the male luer and female luer rotate to a position in which the second vent passage is aligned with the slot along the longitudinal axis of the vacuum luer lock connector, air rushes in to the vacuum chamber and the vacuum seal dissipates, allowing the male luer and female luer to disconnect easily. In some embodiments, a tip of the male luer separates the vacuum chamber from the fluid passageway. In some embodiments, the vacuum luer lock connector further comprises a designation “LOCK” to inform a user if the second vent passage and the slot are not aligned and the vacuum luer lock connector is being held connected. In some embodiments, the vacuum luer lock connector further comprises a designation “UNLOCK” to inform a user if the second vent passage and the slot are aligned and the vacuum luer lock connector is not being held connected. In some embodiments, the male luer includes a vent passage. In some embodiments, the female luer further comprises a second cylindrical member that separates the fluid passageway from the vacuum chamber when the male luer mates with the female luer.

Embodiments of the present disclosure provide a method for using a vacuum luer lock connector comprising connecting a male luer and a female luer to form a vacuum seal and define a fluid passageway, the vacuum luer lock comprising a vacuum chamber between the male luer and female luer that entraps air when the male luer is pushed into the female luer, wherein the male luer and female luer are held connected by a force created in the vacuum chamber that surrounds the male luer and female luer. In some embodiments, the vacuum luer lock further comprises a solid cylinder that surrounds the male luer and acts as a piston a cylindrical member that surrounds the female luer and acts as a wall of the vacuum chamber, and a vent passage in the female luer that allows air to escape when the male luer is pushed into the female luer. In some embodiments, the vacuum luer lock further comprises a second vent passage in the piston of the male luer that connects a compression area of the male luer to a surface of the male luer that mates with the female luer, the surface being perpendicular to a longitudinal axis of the male luer. In some embodiments, the vacuum luer lock further comprises a slot created on an inside surface of the cylindrical member, the slot extending from an edge of the cylindrical member that mates with the male luer in a direction away from the male luer along a longitudinal axis of the solid cylinder. In some embodiments, the method further comprises rotating the male luer with respect to the female luer such that the second vent passage and the slot are not aligned with one another along a longitudinal axis of the vacuum luer lock connector to maintain sufficient vacuum seal. In some embodiments, the method further comprises rotating the male luer with respect to the female luer to a position in which the second vent passage is aligned with the slot along the longitudinal axis of the vacuum luer lock connector to allow air to rush into the vacuum chamber and the vacuum seal to dissipate, and disconnecting the male luer and female luer.

It is to be understood that both the foregoing general description and the following detailed description are exemplary and explanatory and are intended to provide further explanation of the subject technology as claimed. It is also to be understood that other aspects may be utilized, and changes may be made without departing from the scope of the subject technology.

### BRIEF DESCRIPTION OF THE DRAWINGS

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

FIG. 1 illustrates an IV administration set coupled to a patient, in accordance with aspects of the present disclosure.

FIG. 2 is a cross sectional view of a vacuum luer lock connector in accordance with some embodiments of the present disclosure.

FIGS. 3A-3B are perspective views of a vacuum luer lock connector in accordance with some embodiments of the present disclosure.

FIG. 4 is a cross sectional view of a vacuum luer lock connector in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

The present description relates in general to infusion fluid lines connectors, and more particularly, for example and without limitation, to more particularly to vacuum luer lock connectors held together with a vacuum seal. In accordance with some embodiments, the vacuum luer lock connector may be a two-piece assembly, including a male luer and a female luer.

In some embodiments, the vacuum luer lock connector is used in an IV administration set having a structure for coupling with an IV bag and/or a drip chamber. The structure for the IV bag can be formed as an arm, hook, clamp, or another mechanism configured to suspend the IV bag.

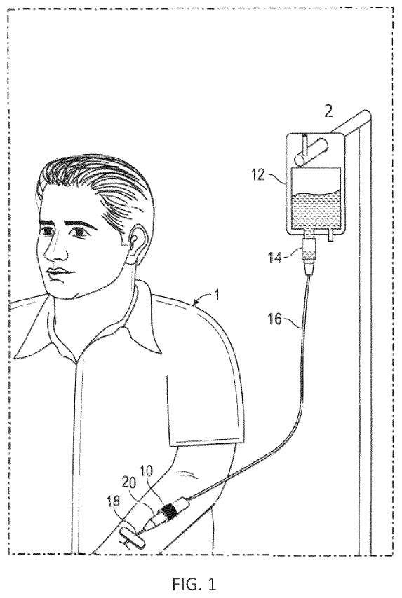

Referring to FIGS. 1, an embodiment of an IV administration set 2 is coupled to a patient 1. The IV administration set 2 may include a vacuum luer lock connector 100. The IV administration set includes an IV bag 12 and a drip chamber 14 fluidly coupled together by IV tubing 16 extending therebetween. The IV tubing 16 is intravenously coupled to the patient 1 through a catheter 18, and the catheter preferably includes one or more adapters 10 or connectors 20.

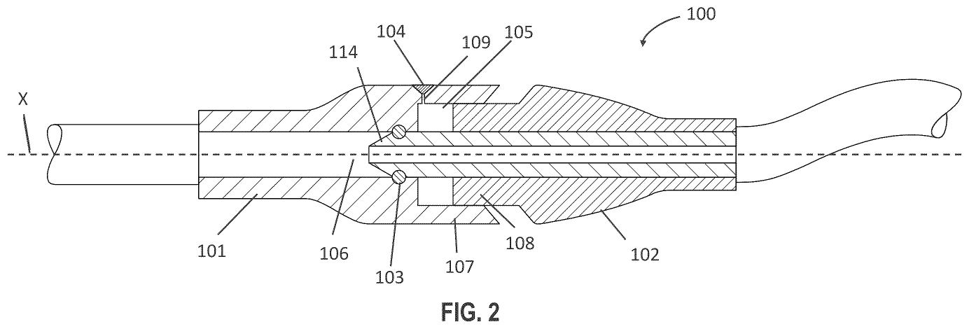

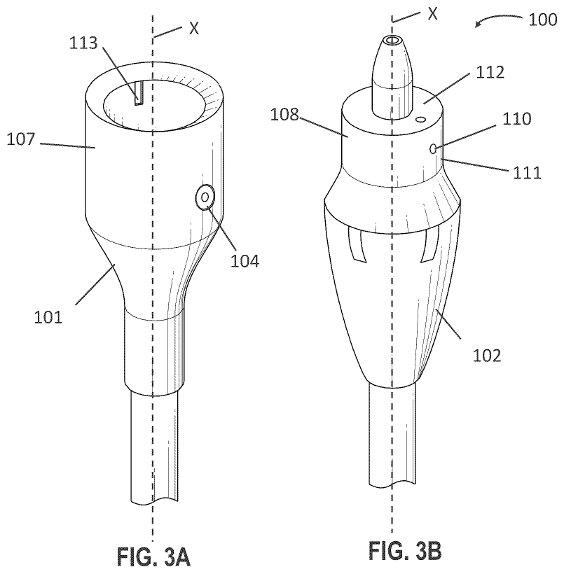

Referring to FIG. 2-3B, an embodiment of a vacuum luer lock connector 100 is shown comprising a male luer 102 configured to couple to a female luer 101 defining a fluid passageway 106, a vacuum chamber 105 between the male luer 102 and female luer 101 that entraps air when the male luer 102 is pushed into the female luer 101, a solid cylinder 108 that surrounds the male luer 102 and acts as a piston, a cylindrical member 107 that surrounds the female luer 101 and acts as a wall of the vacuum chamber 105, and a vent passage 109 in the female luer 101 that allows air to escape when the male luer 102 is pushed into the female luer 101. The male luer 102 and female luer 101 are held connected by a force created in the vacuum chamber 105 that surrounds the luers. In some embodiments, the vacuum luer lock connector 100 comprises an o-ring 103 or a plurality of o-rings 103 installed inside the female luer 101 that prevents an infusion fluid from leaking into the vacuum chamber 105. In some embodiments, the vacuum luer lock connector 100 comprises a check valve 104 on an outer surface of the vent passage 109 that prevents air from flowing back into the vacuum chamber 105. The check valve 104 allows air or gas to be evacuated from the vacuum chamber 105 when the male luer 102 and female luer 101 are connected. In some embodiments, the check valve 104 can function as a one-way valve to permit air to be evacuated from the vacuum chamber 105 and restrict or prevent air from entering into the vacuum chamber 105 after the luers 102, 101 are connected.

In some embodiments, the vacuum luer lock connector 100 comprises a second vent passage 110 in the solid cylinder 108 of the male luer 102 that connects the compression area 111 of the male luer 102 to a surface 112 of the male luer 102 that mates with the female luer 101, the surface 112 being perpendicular to a longitudinal axis X of the male luer 102. In some embodiments, a slot 113 is created on an inside surface of the cylindrical member 107. In some embodiments, the slot 113 extends from the edge of the cylindrical member 107 of the female luer 101 that mates with the male luer 102 in a direction away from the male luer 102 along a longitudinal axis X of the cylindrical member 107. In some embodiments, the second vent passage 110 and the slot 113 should not be aligned with one another along a longitudinal axis of the connector to maintain a sufficient vacuum seal. In some embodiments, when the male luer 102 and female luer 101 rotate to a position in which the second vent passage 110 is aligned with the slot 113 along the longitudinal axis of the vacuum luer lock connector 100, air rushes into the vacuum chamber 105 and the vacuum seal dissipates, allowing the male luer 102 and female luer 101 to disconnect easily. In some embodiments, a tip 114 of the male luer 102 separates the vacuum chamber 105 from the fluid passageway 106. In some embodiments, the vacuum luer lock connector 100 comprises a designation “LOCK” to inform a user if the second vent passage 110 and the slot 113 are not aligned and the vacuum luer lock connector 100 is being held connected. In some embodiments, the vacuum luer lock connector 100 comprises a designation “UNLOCK” to inform a user if the second vent passage 110 and the slot 113 are aligned and the vacuum luer lock connector 100 is not being held connected. The vacuum force is strong and controllable and it does not rely on high contact force to hold and seal the connections. Also, the use of the o-rings 103 prevents the contacting surfaces from liquid exposure and creates sufficient sealing pressure over a significantly smaller surface area. This combination allows a secure and strong connection along with ease of disconnection.

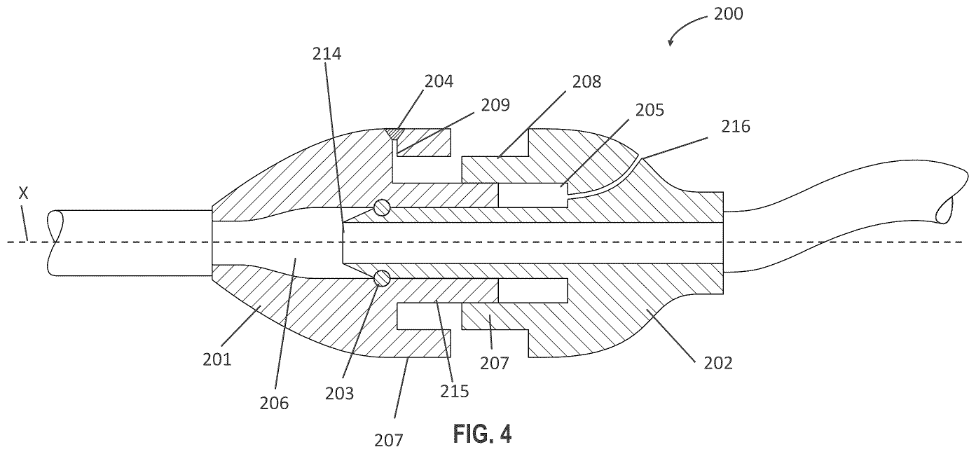

Referring to FIG. 4 an embodiment of a vacuum luer lock connector 200 is shown. comprising a male luer 202 configured to couple to a female luer 201 defining a fluid passageway 206, a vacuum chamber 205 between the male luer 202 and female luer 201 that entraps air when the male luer 202 is pushed into the female luer 201, a solid cylinder 208 that surrounds the male luer 202 and acts as a piston, a cylindrical member 207 that surrounds the female luer 201 and acts as the walls of the vacuum chamber 205, a second cylindrical member 215 that separates the fluid passageway 206 from the vacuum chamber 205 to protect against fluid exposure and contamination, and a vent passage 209 in the female luer 201 that allows air to escape when the male luer 202 is pushed into the female luer 201. In some embodiments, the male luer 202 also includes a vent passage 216. The male luer 202 and female luer 201 are held connected by a force created in the vacuum chamber 205 that surrounds the luers. In some embodiments, the vacuum luer lock connector 200 comprises an o-ring 203 or a plurality of o-rings 203 installed inside the female luer 201 that prevents an infusion fluid from leaking into the vacuum chamber 205. In some embodiments, the vacuum luer lock connector 200 comprises a check valve 204 on an outer surface of the vent passage 109 that prevents air from flowing back into the vacuum chamber 205.

In some embodiments, the vacuum luer lock connector 200 comprises a second vent passage 216 in the male luer 202 that connects the vacuum chamber to the exterior of the connector. In some embodiments, this second vent passage 216 includes a check valve. The vacuum force is strong and controllable and it does not rely on high contact force to hold and seal the connections. Also, the use of the o-rings 203 prevents the contacting surfaces from liquid exposure and creates sufficient sealing pressure over a significantly smaller surface area. This combination allows a secure and strong connection along with ease of disconnection.

Some embodiments of the present disclosure provide a method for using a vacuum luer lock in accordance with any of the embodiments described above. In some embodiments, the method includes connecting a male luer and a female luer to form a vacuum seal and define a fluid passageway. In some embodiments, the method includes rotating the male luer with respect to the female luer such that the second vent passage and the slot are not aligned with one another along a longitudinal axis of the connector to maintain sufficient vacuum. In some embodiments, the method includes rotating the male luer with respect to the female luer to a position in which the second vent passage is aligned with the slot along the longitudinal axis of the connector to allow air to rush into the vacuum chamber and the vacuum seal to dissipate; and disconnecting the connectors.

In some embodiments, the method includes controlling a check valve with a user's finger from outside of the vacuum luer lock connecter to allow air to rush in for disconnection. In some embodiments, the method includes controlling the incoming and outgoing air by a single vent line equipped with a controllable check valve.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 9. The other clauses can be presented in a similar manner.

Clause 1. A vacuum luer lock connector, comprising a male luer configured to couple to a female luer defining a fluid passageway, a vacuum chamber between the male luer and female luer that entraps air when the male luer is pushed into the female luer, wherein the male luer and female luer are held connected by a force created in the vacuum chamber that surrounds the male luer and female luer.

Clause 2. The vacuum luer lock connector of Clause 1 further comprising a vent passage in the female luer that allows air to escape when the male luer is pushed into the female luer.

Clause 3. The vacuum luer lock connector of Clause 1 further comprising a piston, wherein the piston is a solid cylinder that surrounds the male luer.

Clause 4. The vacuum luer lock connector of Clause 3 further comprising a cylindrical member that surrounds the female luer and acts as a wall of the vacuum chamber.

Clause 5. The vacuum luer lock connector of Clause 1 further comprising an o-ring installed inside the female luer that prevents an infusion fluid from leaking into the vacuum chamber.

Clause 6. The vacuum luer lock connector of Clause 5, wherein a plurality of o-rings are installed inside the female luer.

Clause 7. The vacuum luer lock connector of Clause 2, further comprising a check valve on an outer surface of the vent passage that prevent the air from flowing back into the vacuum chamber.

Clause 8. The vacuum luer lock connector of Clause 4 further comprising a second vent passage in the piston of the male luer that connects a compression area of the male luer to a surface of the male luer that mates with the female luer, the surface being perpendicular to a longitudinal axis of the male luer, and a slot created on an inside surface of the cylindrical member, the slot extending from an edge of the cylindrical member that mates with the male luer in a direction away from the male luer along a longitudinal axis of the solid cylinder, wherein a vacuum seal is maintained when the second vent passage and the slot are not aligned with one another along a longitudinal axis of the vacuum luer lock connector.

Clause 9. The vacuum luer lock connector of Clause 8, wherein when the male luer and female luer rotate to a position in which the second vent passage is aligned with the slot along the longitudinal axis of the vacuum luer lock connector, air rushes in to the vacuum chamber and the vacuum seal dissipates, allowing the male luer and female luer to disconnect easily.

Clause 10. The vacuum luer lock connector of Clause 1, wherein a tip of the male luer separates the vacuum chamber from the fluid passageway.

Clause 11. The vacuum luer lock connector of Clause 8 further comprising a designation “LOCK” to inform a user if the second vent passage and the slot are not aligned and the vacuum luer lock connector is being held connected.

Clause 12. The vacuum luer lock connector of Clause 8 further comprising a designation “UNLOCK” to inform a user if the second vent passage and the slot are aligned and the vacuum luer lock connector is not being held connected.

Clause 13. The vacuum luer lock connector of Clause 1, wherein the male luer includes a vent passage.

Clause 14. The vacuum luer lock connector of Clause 1, wherein the female luer further comprises a second cylindrical member that separates the fluid passageway from the vacuum chamber when the male luer mates with the female luer.

Clause 15. A method for using a vacuum luer lock connector comprising connecting a male luer and a female luer to form a vacuum seal and define a fluid passageway, the vacuum luer lock comprising a vacuum chamber between the male luer and female luer that entraps air when the male luer is pushed into the female luer, wherein the male luer and female luer are held connected by a force created in the vacuum chamber that surrounds the male luer and female luer.

Clause 16. The method for using the vacuum luer lock connector of Clause 15, wherein the vacuum luer lock further comprises a solid cylinder that surrounds the male luer and acts as a piston a cylindrical member that surrounds the female luer and acts as a wall of the vacuum chamber, and a vent passage in the female luer that allows air to escape when the male luer is pushed into the female luer.

Clause 17. The method for using the vacuum luer lock connector of Clause 16, wherein the vacuum luer lock further comprises a second vent passage in the piston of the male luer that connects a compression area of the male luer to a surface of the male luer that mates with the female luer, the surface being perpendicular to a longitudinal axis of the male luer.

Clause 18. The method for using the vacuum luer lock connector of Clause 17, wherein the vacuum luer lock further comprises a slot created on an inside surface of the cylindrical member, the slot extending from an edge of the cylindrical member that mates with the male luer in a direction away from the male luer along a longitudinal axis of the solid cylinder.

Clause 19. The method for using the vacuum luer lock connector of Clause 18 further comprising rotating the male luer with respect to the female luer such that the second vent passage and the slot are not aligned with one another along a longitudinal axis of the vacuum luer lock connector to maintain sufficient vacuum seal.

Clause 20. The method for using the vacuum luer lock connector of Clause 18 further comprising rotating the male luer with respect to the female luer to a position in which the second vent passage is aligned with the slot along the longitudinal axis of the vacuum luer lock connector to allow air to rush into the vacuum chamber and the vacuum seal to dissipate and disconnecting the male luer and female luer.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

1. A vacuum luer lock connector, comprising:
a male luer configured to couple to a female luer defining a fluid passageway;
a vacuum chamber between the male luer and female luer that entraps air when the male luer is pushed into the female luer;
wherein the male luer and female luer are held connected by a force created in the vacuum chamber that surrounds the male luer and female luer.

2. The vacuum luer lock connector of claim 1 further comprising a vent passage in the female luer that allows air to escape when the male luer is pushed into the female luer.

3. The vacuum luer lock connector of claim 1 further comprising a piston, wherein the piston is a solid cylinder that surrounds the male luer.

4. The vacuum luer lock connector of claim 3 further comprising a cylindrical member that surrounds the female luer and acts as a wall of the vacuum chamber.

5. The vacuum luer lock connector of claim 1 further comprising an o-ring installed inside the female luer that prevents an infusion fluid from leaking into the vacuum chamber.

6. The vacuum luer lock connector of claim 5, wherein a plurality of o-rings are installed inside the female luer.

7. The vacuum luer lock connector of claim 2, further comprising a check valve on an outer surface of the vent passage that prevent the air from flowing back into the vacuum chamber.

8. The vacuum luer lock connector of claim 4 further comprising:
a second vent passage in the piston of the male luer that connects a compression area of the male luer to a surface of the male luer that mates with the female luer, the surface being perpendicular to a longitudinal axis of the male luer; and
a slot created on an inside surface of the cylindrical member, the slot extending from an edge of the cylindrical member that mates with the male luer in a direction away from the male luer along a longitudinal axis of the solid cylinder,
wherein a vacuum seal is maintained when the second vent passage and the slot are not aligned with one another along a longitudinal axis of the vacuum luer lock connector.

9. The vacuum luer lock connector of claim 8, wherein when the male luer and female luer rotate to a position in which the second vent passage is aligned with the slot along the longitudinal axis of the vacuum luer lock connector, air rushes in to the vacuum chamber and the vacuum seal dissipates, allowing the male luer and female luer to disconnect easily.

10. The vacuum luer lock connector of claim 1, wherein a tip of the male luer separates the vacuum chamber from the fluid passageway.

11. The vacuum luer lock connector of claim 8 further comprising a designation “LOCK” to inform a user if the second vent passage and the slot are not aligned and the vacuum luer lock connector is being held connected.

12. The vacuum luer lock connector of claim 8 further comprising a designation “UNLOCK” to inform a user if the second vent passage and the slot are aligned and the vacuum luer lock connector is not being held connected.

13. The vacuum luer lock connector of claim 1, wherein the male luer includes a vent passage.

14. The vacuum luer lock connector of claim 1, wherein the female luer further comprises a second cylindrical member that separates the fluid passageway from the vacuum chamber when the male luer mates with the female luer.

15. A method for using a vacuum luer lock connector comprising:
connecting a male luer and a female luer to form a vacuum seal and define a fluid passageway, the vacuum luer lock comprising:
a vacuum chamber between the male luer and female luer that entraps air when the male luer is pushed into the female luer;
wherein the male luer and female luer are held connected by a force created in the vacuum chamber that surrounds the male luer and female luer.

16. The method for using the vacuum luer lock connector of claim 15, wherein the vacuum luer lock further comprises a solid cylinder that surrounds the male luer and acts as a piston;
a cylindrical member that surrounds the female luer and acts as a wall of the vacuum chamber; and
a vent passage in the female luer that allows air to escape when the male luer is pushed into the female luer.

17. The method for using the vacuum luer lock connector of claim 16, wherein the vacuum luer lock further comprises a second vent passage in the piston of the male luer that connects a compression area of the male luer to a surface of the male luer that mates with the female luer, the surface being perpendicular to a longitudinal axis of the male luer.

18. The method for using the vacuum luer lock connector of claim 17, wherein the vacuum luer lock further comprises a slot created on an inside surface of the cylindrical member, the slot extending from an edge of the cylindrical member that mates with the male luer in a direction away from the male luer along a longitudinal axis of the solid cylinder.

19. The method for using the vacuum luer lock connector of claim 18 further comprising:
rotating the male luer with respect to the female luer such that the second vent passage and the slot are not aligned with one another along a longitudinal axis of the vacuum luer lock connector to maintain sufficient vacuum seal.

20. The method for using the vacuum luer lock connector of claim 18 further comprising:
rotating the male luer with respect to the female luer to a position in which the second vent passage is aligned with the slot along the longitudinal axis of the vacuum luer lock connector to allow air to rush into the vacuum chamber and the vacuum seal to dissipate; and
disconnecting the male luer and female luer.
